(19) United States
(12) Patent Application Publication
(10) Pub. No.: US 2009/0252757 A1
(43) Pub. Date: Oct. 8, 2009

(54) METHODS AND COMPOSITIONS FOR INHIBITING HIV INFECTION

(75) Inventors: Deborah Nguyen, San Dieago, CA (US); Kelli L. Kuhen, Solana Beach, CA (US); Jeremy S. Caldwell, La Jolla, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/095,867

(22) PCT Filed: Dec. 8, 2006
(86) PCT No.: PCT/US2006/046866

§ 371 Date: Jun. 2, 2008

Related U.S. Application Data
(60) Provisional application No. 60/748,759, filed on Dec. 8, 2005.

(51) Int. Cl. A61K 36/06; C12Q 1/70; A61K 31/553; A61P 31/18; A61K 31/095

(52) U.S. Cl. 424/195.15; 435/5; 514/211.08; 514/706

(57) ABSTRACT

This invention provides novel HIV-interacting host factors. The invention also provides methods of using the HIV-interacting host factors to screen for compounds that inhibit HIV infection. The methods comprise first screening test compounds for modulators of an HIV interacting host factor disclosed herein, and then further screening the identified modulating compounds for ability to inhibit HIV infection. The invention further provides methods and pharmaceutical compositions for treating diseases and conditions associated with HIV infection.

### CROSS-REFERENCE TO RELATED APPLICATIONS

This application claims the benefit of priority under 35 U.S.C. §119(e) to U.S. Provisional Patent Application No. 60/748,759 filed Dec. 8, 2005. The disclosure of the priority application is incorporated herein by reference in its entirety and for all purposes.

### FIELD OF THE INVENTION

The present invention generally relates to inhibition of HIV infections. More particularly, the invention pertains to identification of novel HIV-interacting host factors, and to methods of using such host factors to identify novel compounds that inhibit HIV infection.

### BACKGROUND OF THE INVENTION

Human immunodeficiency viruses (HIV) are lentiviruses from the family of retroviridae. It was estimated that transmission of HIV through sexual contact and during pregnancy accounts for up to 90% of AIDS cases worldwide. This transmission is initiated by the passage of the virus across the mucosal barrier of sexual organs or placenta when exposed to infectious body fluids such as semen, vaginal secretions, or blood. The remaining AIDS cases are due to the transfusion of HIV-contaminated blood, needle sharing among intravenous drug users, accidental exposure to HIV-contaminated body fluids during invasive procedures, and other situations wherein infectious virus can come into direct contact with susceptible human tissues.

The currently available drugs for treating HIV infection and AIDS are not satisfactory. Toxicity or undesirable side effects of the common drugs for treating HIV infection, e.g., AZT or HIV protease inhibitors, are incompatible with their antiviral activity when used at an effective pharmaceutical concentration. Due to the high mutability of the viral genome, resistance to the currently available drugs is rapidly generated and then spread throughout the population. Thus, there is still a need in the art for better alternative compounds and novel therapeutic targets for preventing and treating AIDS and HIV infection. The instant invention addresses this and other needs.

### SUMMARY OF THE INVENTION

In one aspect, the invention provides methods for identifying agents that inhibit HIV infection. The methods involve screening test compounds to identify one or more modulating compounds that down-regulate a biological activity or expression level of an HIV-interacting host factor encoded by a polynucleotide selected from the members listed in Tables 2-4, and then testing the identified modulating compounds for ability to inhibit HIV infection. In some of the methods, the HIV-interacting host factor employed is tetratricopeptide repeats 1 (IFIT1), phosphatidylinositol transfer protein alpha (PITPNα), or mixed lineage kinase 3 (MLK3). Some of these methods employ MLK3, and the test compounds are screened for ability to inhibit the kinase activity of MLK3 or its expression.

In some of the methods, the ability to inhibit HIV infection by the modulating compounds is examined by monitoring expression of a reporter gene under the control of HIV LTR promoter in an HIV-infected cell. For example, the HIV-infected cell can be a HeLa-CD4-Bgal cell which expresses the beta-galactosidase reporter gene. In some of these methods, the cell is infected by the HIV-IIIb virus strain.

In some other methods, the ability to inhibit HIV-1 infection by a modulating compound is examined by comparing HIV replication in an engineered HIV permissive cell that has been contacted with the modulating compound with HIV replication in a control cell that has not been contacted with the compound. In some methods, the HIV permissive cell employed is HeLa-T4-βGal HIV cell. In some methods, HIV replication is monitored via a p24 antigen ELISA assay or a reverse transcriptase activity assay.

In some other methods, the ability to inhibit HIV infection by the compound is examined by comparing pseudovirus production in a host cell treated with the compound with pseudovirus production in a control host cell that has not been treated with the compound. In some of these methods, the host cell used is 293T HEK cell. In some of these methods, the host cell is transfected with pseudovirus plasmids which produce HIV pseudovirus in the cell.

Some of the screening methods employ an HIV-interacting host factor that is an enzyme. In these methods, the biological activity assayed is typically its enzymatic activity. Some of these methods employ an HIV-interacting host factor that is a kinase, e.g., MLK3.

In another aspect, the invention provides methods for inhibiting HIV infection in a subject. These methods involve administering to the subject a pharmaceutical composition which comprises an effective amount of a compound that inhibits a biological activity or expression of an HIV-interacting host factor. The HIV-interacting factor is encoded by a polynucleotide selected from the members listed in Tables 2-4. Some of the methods employ a compound that inhibits a biological activity or expression of tetratricopeptide repeats 1 (IFIT1), phosphatidylinositol transfer protein alpha (PITPNα), or mixed lineage kinase 3 (MLK3). In some of these methods, the therapeutic compound employed inhibits the kinase activity of MLK3, e.g., K252a or CEP1347.

A further understanding of the nature and advantages of the present invention may be realized by reference to the remaining portions of the specification and claims.

### BRIEF DESCRIPTION OF THE DRAWINGS

FIG. 1 shows effect of kinase-inactive MLK3 (K144R) on HIV infection of HeLaCD4βgal cells. Cells were co-transfected with cDNA encoding wild type MLK3, kinase-inactive MLK3 (K144R), or control plasmid along with a luciferase reporter under the control of the HIV-LTR followed after 24 hours by infection with HIV-IIIb. Infection was assessed after 3 days by measuring luciferase activity using Brite Glo. The kinase-inactive mutant was unable to increase infection, highlighting the requirement of kinase function for the enhancement seen by wild type MLK3. Data is shown as the fold enhancement over negative control plasmid and is the summary of four independent assays with two replicates in each assay.

FIG. 2 shows effect of MLK3 overexpression on HIV transcription. HeLaCD4βgal cells were co-transfected with cDNA encoding wild type MLK3 or control plasmid (pcDNA3) along with a luciferase reporter under the control of the HIV-LTR (LTRLuc) and either a Tat expression vector (Tat) or control vector Sport6-GFP(S6G) to assess either Tat-dependent or Tat-independent transcription. Transcription was assessed after 2 days by measuring luciferase activity using Brite Glo. Expression of MLK3 enhanced Tat-dependent transcription of luciferase, but had minimal effect on Tat-independent transcription. Data is shown as the average fold enhancement with MLK3 (e.g., MLK3/Tat/LTRLuc) over negative control plasmid (e.g., pcDNA3/Tat/LTRLuc), and is the summary of two independent assays with two replicates in each assay.

FIGS. 3A-3D show efficacy of siRNA targeting MLK3 against HIV infection. A: Effect on HIV infection by individual MLK3 siRNAs transfected into HeLaCD4βgal cells; B: Significant depletion of endogenous MLK3 level in HeLaCD4βgal cells by the siRNAs targeting MLK3; C: Effect on HIV infection by individual MLK3 siRNAs electroporated into Jurkat cells; and D: Depletion of endogenous MLK3 level in Jurkat cells by the siRNAs targeting MLK3. Data is shown as the percent infection inhibition or cytotoxicity compared to control GL2 siRNA deviation and is the average +/− standard deviation of at least three independent experiments with twelve replicate wells per experiment.

### DETAILED DESCRIPTION

### I. Overview

The invention is predicated in part on the discoveries by the present inventors of novel host factors involved in HIV infection. As detailed in Examples below, the present inventors screened both a focused siRNA library (Qiagen) and a cDNA library representing 15,000 unique genes (Origene) using HeLa-CD4-βgal cells and HIV-IIIb. 96 genes from the siRNA screening hits whose knockdown inhibited HIV infection were chosen for follow-up studies. These include the two known HIV-interacting host factors, Furin and Rad23. Another hit identified from the screening is Pak3, which was disclosed in U.S. Provisional Patent Application No. 60/650,789. Nearly all the siRNA hits selected reconfirmed in the original assay, however 62.5% had severe cytotoxicity. From the original 96 hits, 36 reconfirmed and exhibited efficacy against HIV that was much greater than their cytotoxic effects in HeLa-CD4-βgal cells. These hits are shown in Table 2. From the cDNA screen, 89 enhancers of HIV infection were chosen for follow-up, which included testing of additional preparations of the cDNAs and sequence confirmation. Of the 89 hits, 13 continued to show enhancement of HIV infection throughout reconfirmation. These confirmed hits are shown in Table 3.

Additional host factors that could play a role in HIV infection are also identified by the present inventors from a yeast two-hybrid screen. As detailed in Example 4 below, the present inventors screened a human leukocyte cDNA library cloned into the GAL4 expression vector (Clontech) to identify novel interacting host cell factors, using HIV-1 HXB2 Vpr as the bait. Vpr is an HIV accessory protein which is essential for viral replication in monocytes and macrophages, and increases viral replication in T cells and T cell lines. Positive colonies from the screening were assayed for β-galactosidase activity using a filter lift assay for further confirmation of the protein-protein interaction. One Vpr interacting partner identified from the screening is isopeptidase T (IsoT), disclosed in more detail in U.S. Provisional Patent Application No. 60/673,623. The other screen hits are shown in Table 4 herein.

The host molecules identified through siRNA screening, cDNA screening, and yeast two hybrid screening are termed herein “HIV-interacting host factors.” These host factors could play important roles in various stages of HIV infection. They also provide novel targets which can be used to screen for compounds that inhibit HIV infections. The following sections provide further guidance for employing these host factors to identify novel anti-HIV agents.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Oxford Dictionary of Biochemistry and Molecular Biology, Smith et al. (eds.), Oxford University Press (revised ed., 2000); Dictionary of Microbiology and Molecular Biology, Singleton et al. (Eds.), John Wiley & Sons (3^{rd} ed., 2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more molecules (e.g., a test agent and a polypeptide) or combining molecules and cells (e.g., a test agent and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

A “heterologous sequence” or a “heterologous polynucleotide,” as used herein, is one that originates from a source foreign to the particular host cell, or, if from the same source, is modified from its original form. Thus, a heterologous polynucleotide in a host cell includes a polynucleotide that, although being endogenous to the particular host cell, has been modified. Modification of the heterologous sequence can occur, e.g., by treating the polynucleotide with a restriction enzyme to generate a polynucleotide fragment that is capable of being operably linked to the promoter. Techniques such as site-directed mutagenesis are also useful for modifying a heterologous polynucleotide.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology.

A “host cell,” as used herein, refers to a prokaryotic or eukaryotic cell into which a heterologous polynucleotide (e.g., an expression vector) is to be introduced. The heterologous polynucleotide can be introduced into the host cell by any means, e.g., transfection, electroporation, calcium phosphate precipitation, microinjection, transformation, viral infection, and/or the like.

The term “HIV-interacting host factor” refers to host genes or their encoded polypeptides which are identified by the present inventors to play a role in facilitating HIV infection or life cycle. As shown in Tables 2-4, these factors include host molecules whose knockdown leads to suppression of HIV replication and also molecules whose overexpression results in enhanced HIV infection. In addition, the term also encompasses host factors which physically interact with HIV accessory protein Vpr which is essential for HIV viral infection.

The term “sequence identity” in the context of two nucleic acid sequences or amino acid sequences refers to the residues in the two sequences which are the same when aligned for maximum correspondence over a specified comparison window. A “comparison window” refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci. U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). Alignment can also be performed by inspection and manual alignment.

A “substantially identical” nucleic acid or amino acid sequence refers to a nucleic acid or amino acid sequence which has at least 90% sequence identity to a reference sequence using the programs described above (e.g., BLAST) using standard parameters. The sequence identity is preferably at least 95%, more preferably at least 98%, and most preferably at least 99%. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “modulate” with respect to a biological activity of a reference protein or its fragment refers to a change in the expression level or other biological activities of the protein. For example, modulation may cause an increase or a decrease in expression level of the reference protein, enzymatic modification (e.g., phosphorylation) of the protein, binding characteristics (e.g., binding to a target polynucleotide), or any other biological, functional, or immunological properties of the reference protein. The change in activity can arise from, for example, an increase or decrease in expression of one or more genes that encode the reference protein, the stability of an mRNA that encodes the protein, translation efficiency, or from a change in other biological activities of the reference protein. The change can also be due to the activity of another molecule that modulates the reference protein (e.g., a kinase which phosphorylates the reference protein). Modulation of a reference protein can be up-regulation (i.e., activation or stimulation) or down-regulation (i.e. inhibition or suppression). The mode of action of a modulator of the reference protein can be direct, e.g., through binding to the protein or to genes encoding the protein, or indirect, e.g., through binding to and/or modifying (e.g., enzymatically) another molecule which otherwise modulates the reference protein.

The term “subject” includes mammals, especially humans. It also encompasses other non-human animals such as cows, horses, sheep, pigs, cats, dogs, mice, rats, rabbits, guinea pigs, monkeys.

A “variant” of a reference molecule refers to a molecule substantially similar in structure and biological activity to either the entire reference molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

### III. Screening for Novel Modulators of HIV Infections—General Scheme

The HIV-interacting host factors identified by the present inventors provide novel targets to screen for compounds that inhibit HIV infections. Various biochemical and molecular biology techniques or assays well known in the art can be employed to practice the present invention. Such techniques are described in, e.g., Handbook of Drug Screening, Seethala et al. (eds.), Marcel Dekker (1^{st} ed., 2001); High Throughput Screening Methods and Protocols (Methods in Molecular Biology, 190), Janzen (ed.), Humana Press (1^{st} ed., 2002); Current Protocols in Immunology, Coligan et al. (Ed.), John Wiley & Sons Inc (2002); Sambrook et al., Molecular Cloning: A Laboratory Manual, Cold Spring Harbor Press (3^{rd} ed., 2001); and Brent et al., Current Protocols in Molecular Biology, John Wiley & Sons, Inc. (ringbou ed., 2003).

Typically, test agents are first screened for their ability to modulate a biological activity of an HIV-interacting host factor encoded by the polynucleotides shown in Tables 2-4 (“the first assay step”). Modulating agents thus identified are then subject to further screening for ability to inhibit HIV infection, typically in the presence of the HIV-interacting host factor (“the second testing step”). Depending on the HIV-interacting host factor employed in the method, modulation of different biological activities of the HIV-interacting host factor can be assayed in the first step. For example, a test agent can be assayed for binding to the HIV-interacting host factor. The test agent can be assayed for activity to modulate expression of the HIV-interacting host factor, e.g., transcription or translation. The test agent can also be assayed for activities in modulating expression or cellular level of the HIV-interacting host factor, e.g., post-translational modification or proteolysis.

Test agents can be screened for ability to either up-regulate or down-regulate a biological activity of the HIV-interacting host factor in the first assay step. Once test agents that inhibit HIV-interacting host factor are identified, they are typically further tested for ability to inhibit HIV infection. This further testing step is often needed to confirm that their modulatory effect on the HIV-interacting host factor would indeed lead to inhibition of HIV infection. For example, a test agent which inhibits a biological activity of an HIV-interacting host factor shown in Tables 2-4) needs to be further tested in order to confirm that such modulation can result in suppressed or reduced HIV infection.

In both the first assaying step and the second testing step, either an intact HIV-interacting host factor, or a fragment thereof, may be employed. Molecules with sequences that are substantially identical to that of the HIV-interacting host factor can also be employed. Analogs or functional derivatives of the HIV-interacting host factor could similarly be used in the screening. The fragments or analogs that can be employed in these assays usually retain one or more of the biological activities of the HIV-interacting host factor (e.g., kinase activity if the HIV-interacting host factor employed in the first assaying step is a kinase). Fusion proteins containing such fragments or analogs can also be used for the screening of test agents. Functional derivatives of an HIV-interacting host factor usually have amino acid deletions and/or insertions and/or substitutions while maintaining one or more of the bioactivities and therefore can also be used in practicing the screening methods of the present invention. A functional derivative can be prepared from an HIV-interacting host factor by proteolytic cleavage followed by conventional purification procedures known to those skilled in the art. Alternatively, the functional derivative can be produced by recombinant DNA technology by expressing only fragments of an HIV-interacting host factor that retain one or more of their bioactivities.

### IV. Test Compounds

Test agents or compounds that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins. The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecule organic compounds, e.g., chemical compounds with a molecular weight of not more than about 1,000 or not more than about 500. Preferably, high throughput assays are adapted and used to screen for such small molecules. In some methods, combinatorial libraries of small molecule test agents as described above can be readily employed to screen for small molecule compound that inhibit HIV infection. A number of assays are available for such screening, e.g., as described in Schultz (1998) Bioorg Med Chem Lett 8:2409-2414; Weller (1997) Mol Divers. 3:61-70; Fernandes (1998) Curr Opin Chem Biol 2:597-603; and Sittampalam (1997) Curr Opin Chem Biol 1:384-91.

Libraries of test agents to be screened with the claimed methods can also be generated based on structural studies of the HIV-interacting host factors discussed above or their fragments. Such structural studies allow the identification of test agents that are more likely to bind to the HIV-interacting host factors. The three-dimensional structures of the HIV-interacting host factors can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of HIV-interacting host factors' structures provides another means for designing test agents to screen for modulators of HIV infections. Methods of molecular modeling have been described in the literature, e.g., U.S. Pat. No. 5,612,894 entitled “System and method for molecular modeling utilizing a sensitivity factor,” and U.S. Pat. No. 5,583,973 entitled “Molecular modeling method and system.” In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wuthrich (Wiley-Interscience, New York 1986).

Modulators of the present invention also include antibodies that specifically bind to an HIV-interacting host factor in Tables 2-4. Such antibodies can be monoclonal or polyclonal. Such antibodies can be generated using methods well known in the art. For example, the production of non-human monoclonal antibodies, e.g., murine or rat, can be accomplished by, for example, immunizing the animal with an HIV-interacting host factor in Tables 2-4 or its fragment (See Harlow and Lane, Antibodies, A Laboratory Manual, Cold Spring Harbor Press, 3^{rd} ed., 2000). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to an HIV-interacting host factor in Tables 24.

Human antibodies against an HIV-interacting host factor can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using an HIV-interacting host factor or its fragment.

### V. Screening for Modulators of HIV-Interacting Host Factors

Typically, test agents are first screened for ability to modulate a biological activity of an HIV-interacting host factor identified by the present inventors. A number of assay systems can be employed in this screening step. The screening can utilize an in vitro assay system or a cell-based assay system. In this screening step, test agents can be screened for binding to an HIV-interacting host factor, altering expression level of the HIV-interacting host factor, or modulating other biological activities (e.g., enzymatic activities) of the HIV-interacting host factor.

1. Modulating Binding Activities of HIV-Interacting Host Factors

In some methods, binding of a test agent to an HIV-interacting host factor is determined in the first screening step. Binding of test agents to an HIV-interacting host factor can be assayed by a number of methods including e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The test agent can be identified by detecting a direct binding to the HIV-interacting host factor, e.g., co-immunoprecipitation with the HIV-interacting host factor by an antibody directed to the HIV-interacting host factor. The test agent can also be identified by detecting a signal that indicates that the agent binds to the HIV-interacting host factor, e.g., fluorescence quenching or FRET.

Competition assays provide a suitable format for identifying test agents that specifically bind to an HIV-interacting host factor. In such formats, test agents are screened in competition with a compound already known to bind to the HIV-interacting host factor. The known binding compound can be a synthetic compound. It can also be an antibody, which specifically recognizes the HIV-interacting host factor, e.g., a monoclonal antibody directed against the HIV-interacting host factor. If the test agent inhibits binding of the compound known to bind the HIV-interacting host factor, then the test agent also binds the HIV-interacting host factor.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253, 1983); solid phase direct biotin-avidin EIA (see Kirkland et al., J. Immunol, 137:3614-3619, 1986); solid phase direct labeled assay, solid phase direct labeled sandwich assay (see, Harlow and Lane, Antibodies, A Laboratory Manual, Cold Spring Harbor Press, 3^{rd} ed., 2000); solid phase direct label RIA using ¹²⁵I label (see Morel et al., Mol. Immunol. 25(1):7-15, 1988); solid phase direct biotin-avidin EIA (Cheung et al., Virology 176:546-552, 1990); and direct labeled RIA (Moldenhauer et al., Scand. J. Immunol. 32:77-82, 1990). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize an HIV-interacting host factor or its fragment onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the cellular host factor, the test agents are bound to the solid matrix and the HIV-interacting host factor is then added.

Soluble assays include some of the combinatory libraries screening methods described above. Under the soluble assay formats, neither the test agents nor the HIV-interacting host factor are bound to a solid support. Binding of an HIV-interacting host factor or fragment thereof to a test agent can be determined by, e.g., changes in fluorescence of either the HIV-interacting host factor or the test agents, or both. Fluorescence may be intrinsic or conferred by labeling either component with a fluorophor.

In some binding assays, either the HIV-interacting host factor, the test agent, or a third molecule (e.g., an antibody against the HIV-interacting host factor) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group. Similarly, the detectable group can be a substrate, cofactor, inhibitor or affinity ligand.

2. Modulating Other Activities of HIV-Interacting Host Factors

Binding of a test agent to an HIV-interacting host factor provides an indication that the agent can be a modulator of the HIV-interacting host factor. It also suggests that the agent may inhibit HIV infection by acting on the HIV-interacting host factor. Thus, a test agent that binds to an HIV-interacting host factor can be tested for ability to modulate an HIV infection related activity (i.e., in the second testing step outlined above). Alternatively, a test agent that binds to an HIV-interacting host factor can be further examined to determine whether it indeed modulates a biological activity (e.g., an enzymatic activity) of the HIV-interacting host factor. The existence, nature, and extent of such modulation can be tested with an activity assay. More often, such activity assays can be used independently to identify test agents that modulate activities of an HIV-interacting host factor (i.e., without first assaying their ability to bind to the HIV-interacting host factor).

In general, the methods involve adding a test agent to a sample containing an HIV-interacting host factor in the presence or absence of other molecules or reagents which are necessary to test a biological activity of the HIV-interacting host factor (e.g., enzymatic activity if the HIV-interacting host factor is an enzyme), and determining an alteration in the biological activity of the HIV-interacting host factor. If the HIV-interacting host factor has a known biological or enzymatic function (e.g., kinase activity or protease activity), the biological activity monitored in the first screening step can also be the specific biochemical or enzymatic activity of the HIV-interacting host factor. These include kinases (e.g., NTRK1, CCRK, PAK7, MAP3K14, MAPK14, TYK2 and MLK3), proteases (e.g., CTSO), phosphatases (e.g., LOC91443), or other enzymes shown in Tables 2-4 (e.g., NMT1, ALDH3A1, PDE1B and CAT). Any of these molecules can be employed in the first screening step. Methods for assaying the enzymatic activities of these molecules are well known and routinely practiced in the art. The substrates to be used in the screening can be a molecule known to be enzymatically modified by the enzyme (e.g., a kinase), or a molecule that can be easily identified from candidate substrates for a given class of enzymes.

In an exemplary embodiment, the HIV-interacting host factor employed in the screening is the MLK3 kinase, and test agents are first screened for ability to modulate MLK3 kinase activity in autophosphorylation or phosphorylation of a substrate. Effect of test compounds on MLK3 kinase activity can be examined by monitoring MLK3 autophosphorylation in the presence of the compounds using methods as described in, e.g., Gallo et al., J Biol Chem. 269:15092-100, 1994; Leung et al., J Biol Chem. 273:32408-15, 1998; Zhang et al., J. Biol. Chem. 276:45598-603, 2001, and Durkin et al., Biochemistry 43:16348-55, 2004. Compounds inhibiting MLK3 kinase activity can also identified by monitoring phosphorylation of a substrate by MLK3 in the presence of a test compound. For example, the compounds can be examined for effect on MLK3 phosphorylation of golgin-160 in an in vitro assay as described in, e.g., Cha et al., J Cell Sci. 117:751-60, 2004.

Many other assays for monitoring protein kinase activities are also described in the art. These include assays reported in, e.g., Chedid et al., J. Immunol. 147: 867-73, 1991; Kontny et al., Eur J Pharmacol. 227: 333-8, 1992; Wang et al., Oncogene 13: 2639-47, 1996; Murakami et al., Oncogene 14: 243544, 1997; Pyrzynska et al., J. Neurochem. 74: 42-51, 2000; Berry et al., Biochem Pharmacol. 62: 581-91, 2001; Cai et al., Chin Med J (Engl). 114: 248-52, 2001. Any of these methods may be employed and modified to assay modulatory effect of a test agent on an HIV-interacting host factor that is a kinase, e.g., NTRK1, CCRK, PAK7, MAP3K14, MAPK14, TYK2 and MLK3. Further, many kinase substrates are available in the art. See, e.g., www.emdbiosciences.com; and www.proteinkinase.de. In addition, a suitable substrate of a kinase can be screened for in high throughput format. For example, substrates of a kinase can be identified using the Kinase-Glo® luminescent kinase assay (Promega) or other kinase substrate screening kits (e.g., developed by Cell Signaling Technology, Beverly, Mass.).

In addition to assays for screening agents that modulate enzymatic or other biological activities of an HIV-interacting host factor, the activity assays also encompass in vitro screening and in vivo screening for alterations in expression level of the HIV-interacting host factor. Modulation of expression of an HIV-interacting host factor can be examined in a cell-based system by transient or stable transfection of an expression vector into cultured cell lines. For example, test compounds can be assayed for ability to inhibit expression of a reporter gene (e.g., luciferase gene) under the control of a transcription regulatory element (e.g., promoter sequence) of an HIV-interacting host factor. Genes encoding the HIV-interacting host factors shown in Tables 2-4 have all been characterized in the art. Transcription regulatory elements such as promoter sequences of many of these genes have all been delineated.

Assay vector bearing the transcription regulatory element that is operably linked to the reporter gene can be transfected into any mammalian cell line for assays of promoter activity. Reporter genes typically encode polypeptides with an easily assayed enzymatic activity that is naturally absent from the host cell. Typical reporter polypeptides for eukaryotic promoters include, e.g., chloramphenicol acetyltransferase (CAT), firefly or Renilla luciferase, beta-galactosidase, beta-glucuronidase, alkaline phosphatase, and green fluorescent protein (GFP). Vectors expressing a reporter gene under the control of a transcription regulatory element of an HIV-interacting host factor can be prepared using only routinely practiced techniques and methods of molecular biology (see, e.g., e.g., Samrbook et al., supra; Brent et al., supra). In addition to a reporter gene, the vector can also comprise elements necessary for propagation or maintenance in the host cell, and elements such as polyadenylation sequences and transcriptional terminators. Exemplary assay vectors include pGL3 series of vectors (Promega, Madison, Wis.; U.S. Pat. No. 5,670,356), which include a polylinker sequence 5′ of a luciferase gene. General methods of cell culture, transfection, and reporter gene assay have been described in the art, e.g., Samrbook et al., supra; and Transfection Guide, Promega Corporation, Madison, Wis. (1998). Any readily transfectable mammalian cell line may be used to assay expression of the reporter gene from the vector, e.g., HCT116, HEK 293, MCF-7, and HepG2 cells.

### VI. Testing Modulating Compounds for Inhibitory Activity on HIV Infection

To identify novel inhibitors of HIV infection, compounds that modulate an HIV-interacting host factor as described above are typically further tested to confirm their inhibitory effect on HIV infection. Typically, the compounds are screened for ability to modulate an activity that is indicative of HIV infection or HIV replication. The screening is performed in the presence of the HIV-interacting host factor on which the modulating compounds act. The HIV-interacting host factor against which the modulating agents are identified in the first screening step can be either expressed endogenously by the cell or expressed from second expression vector. Preferably, this screening step is performed in vivo using cells that endogenously express the HIV-interacting host factor. As a control, effect of the modulating compounds on a cell that does not express the HIV-interacting host factor may also be examined. For example, if the HIV-interacting host factor (e.g., encoded by a mouse gene) used in the first screening step is not endogenously expressed by the cell line (e.g., a human cell line), a second vector expressing the polypeptide can be introduced into the cell. By comparing an HIV infection related activity in the presence or absence of a modulating compound, activities of the compounds on HIV infection can be identified.

Many assays and methods are available to examine HIV-inhibiting activity of the compounds. This usually involves testing the compounds for ability to inhibit HIV viral replication in vitro or a biochemical activity that is indicative of HIV infection. In some methods, potential inhibitory activity of the modulating compounds on HIV infection can be tested by examining their effect on HIV infection of a cultured cell in vitro, using methods routinely practiced in the art. For example, the compounds can be tested on HIV infection of a primary macrophage culture as described in Seddiki et al., AIDS Res Hum Retroviruses. 15:381-90, 1999. They can also be examined on HIV infection of other T cell and monocyte cell lines as reported in Fujii et al., J Vet Med Sci. 66:115-21, 2004. Additional in vitro systems for monitoring HIV infection have been described in the art. See, e.g., Li et al., Pediatr Res. 54:282-8, 2003; Steinberg et al., Virol. 193:524-7, 1993; Hansen et al., Antiviral Res. 16:233-42, 1991; and Piedimonte et al., AIDS Res Hum Retroviruses. 6:251-60, 1990.

In these assays, HIV infection of the cells can be monitored morphologically, e.g., by a microscopic cytopathic effect assay (see, e.g., Fujii et al., J Vet Med Sci. 66:115-21, 2004). It can also be assessed enzymatically, e.g., by assaying HIV reverse transcriptase (RT) activity in the supernatant of the cell culture. Such assays are described in the art, e.g., Reynolds et al., Proc Natl Acad Sci USA. 100:1615-20, 2003; and Li et al., Pediatr Res. 54:282-8, 2003. Other assays monitor HIV infection by quantifying accumulation of viral nucleic acids or viral antigens. For example, Winters et al. (PCR Methods Appl. 1:257-62, 1992) described a method which assays HIV gag RNA and DNA from HIV infected cell cultures. Vanitharani et al. described an HIV infection assay which measures production of viral p24 antigen (Virology 289:334-42, 2001). Viral replication can also be monitored in vitro by a p24 antigen ELISA assay, as described in, e.g., Chargelegue et al., J Virol Methods. 38(3):323-32, 1992; and Klein et al., J Virol Methods. 107(2):169-75, 2003. All these assays can be employed and modified to assess anti-HIV activity of the modulating compounds of the present invention.

In some methods, potential inhibiting effect of modulating compounds on HIV infection can be examined in engineered reporter cells which are permissive for HIV replication. In these cells, HIV infection and replication is monitored by examining expression of a reporter gene under the control of an HIV transcription regulatory element, e.g., HIV-LTR. One example of such cells is HeLa-T4-βGal HIV reporter cell. As illustrated in Examples below, the HeLa-T4-βGal reporter cell can be infected with HIV-IIIb after being treated with a modulating compound. Virus infectivity from the compound treated cells, as monitored by measuring β-galactosidase activity, can be compared with that from control cells that have not been treated with the compound. A reduced virus titer or reduction in infectivity from cells treated with the modulating compound would confirm that the compound can indeed inhibit HIV infection or viral replication.

In addition to the Hela-T4-βGal cells exemplified herein, many similar reporter assays have also been described in the art. For example, Gervaix et al. (Proc Natl Acad Sci USA. 94:4653-8, 1997) developed a stable T-cell line expressing a plasmid encoding a humanized green fluorescent protein (GFP) under the control of an HIV-1 LTR promoter. Upon infection with HIV-I, a 100- to 1,000-fold increase of fluorescence of infected cells can be observed as compared with uninfected cells. Any of these assay systems can be employed in the present invention to monitor effects of the modulating compounds on HIV infection in real time. These in vitro systems also allow quantitation of infected cells over time and determination of susceptibility to the compounds.

In some other methods, effect of the modulating compounds on HIV replication can be examined by examining production of HIV-1 pseudovirus in a cell treated with the compounds. The cell can express the HIV-interacting host factor endogenously or exogenously. For example, a construct encoding the HIV-interacting host factor can be transfected into the host cell that do not endogenously express the HIV-interacting host factor. As described in more detail in U.S. Provisional Patent Application No. 60/673,623, production of HIV-1 pseudovirus can be obtained by transfecting a producer cell (e.g., a 293T HEK cell) with a reporter plasmid expressing the psi-positive RNA encoding a reporter gene (e.g., luciferase gene), a delta psi packaging construct encoding all structural proteins and the regulatory or accessory proteins such as Tat, Rev, Vpr, and Vif, and a VSV-g envelop expression plasmid. The pseudovirus produced in the producer cell encodes only the reporter gene. After infecting a target cell with pseudovirus in the supernatant from the producer cell, the reporter gene is expressed following retrotranscription and integration into the target cell genome.

To screen for inhibitors of HIV replication, the producer host cell can be treated with a modulating compound prior to, concurrently with, or subsequent to transfection of the pseudovirus plasmids. Preferably, the compound is administered to the host cell prior to transfection of the pseudovirus plasmids, and is present throughout the assay process. Titer of the produced pseudovirus can be monitored by infecting target cells with the pseudovirus in the supernatant from the producer cell and assaying an activity of the reporter (e.g., luciferase activity) in the target cells. As a control, reporter activity in target cells infected with supernatant from producer cells that have not been treated with the compound is also measured. If the modulating compound has an inhibitory effect on virus budding, target cells contacted with the supernatant from the producer cells that have been treated with the compound will have a reduced reporter activity relative to the control cells.

### VII. Therapeutic Applications

By inhibiting HIV infection, the HIV-inhibiting compounds described above provide useful therapeutic applications of the present invention. They can be readily employed to prevent or treat HIV infections, as well as diseases or conditions associated with HIV infections (e.g., AIDS) in various subjects. In addition, compounds known in the art that inhibit any of the HIV-interacting host factors identified by the present inventors can also be used in the therapeutic applications. Examples include K252a and CEP1347 which inhibit the kinase activity of MLK3 (Roux et al., J. Biol. Chem. 277:49473-80, 2002).

HIV infections that are amenable to treatment with the HIV-inhibiting compounds disclosed herein encompass infection of a subject, particularly a human subject, by any of the HIV family of retroviruses (e.g., HIV-I, HIV-II, or HIV-III). The HIV-inhibiting compounds are useful for treating a subject who is a carrier of any member of the HIV family of retroviruses. They can be used to treat a subject who is diagnosed of active AIDS. The compounds are also useful in the treatment or prophylaxis of the AIDS-related conditions in such subjects. Subjects who have not been diagnosed as having HIV infection but are believed to be at risk of infection by HIV are also amenable to treatment with the HIV-inhibiting compounds of the present invention.

Subjects suffering from any of the AIDS-related conditions are suitable for treatment with the HIV-inhibiting compounds. Such conditions include AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

Standard methods for measuring in vivo HIV infection and progression to AIDS can be used to determine whether a subject is positively responding to treatment with the HIV-inhibiting compounds of the invention. For example, after treatment with an HIV-inhibiting compound of the invention, a subject's CD4⁺ T cell count can be monitored. A rise in CD4⁺ T cells indicates that the subject is benefiting from administration of the antiviral therapy. This, as well as other methods known to the art, may be used to determine the extent to which the compounds of the present invention are effective at treating HIV infection and AIDS in a subject.

The HIV-inhibiting compounds of the present invention can be directly administered under sterile conditions to the subject to be treated. The modulators can be administered alone or as the active ingredient of a pharmaceutical composition. The therapeutic composition of the present invention can also be combined with or used in association with other therapeutic agents. In some applications, a first HIV-inhibiting compound is used in combination with a second HIV-inhibiting compound in order to inhibit HIV infection to a more extensive degree than cannot be achieved when one HIV-inhibiting compound is used individually. In some other applications, an HIV-inhibiting compound of the present invention may be used in conjunction with known anti-HIV drugs such as AZT.

Pharmaceutical compositions of the present invention typically comprise at least one active ingredient together with one or more acceptable carriers thereof. Pharmaceutically acceptable carriers enhance or stabilize the composition, or facilitate preparation of the composition. Pharmaceutically acceptable carriers are determined in part by the particular composition being administered (e.g., nucleic acid, protein, or modulatory compounds), as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, intravenous, or parenteral. For example, the HIV-inhibiting compound can be complexed with carrier proteins such as ovalbumin or serum albumin prior to their administration in order to enhance stability or pharmacological properties.

The pharmaceutical compositions can be prepared in various forms, such as granules, tablets, pills, suppositories, capsules, and the like. The concentration of therapeutically active compound in the formulation may vary from about 0.1 100% by weight. Therapeutic formulations are prepared by any methods well known in the art of pharmacy. The therapeutic formulations can be delivered by any effective means which could be used for treatment. See, e.g., Goodman & Gilman's The Pharmacological Bases of Therapeutics, Hardman et al., eds., McGraw-Hill Professional (10^{th} ed., 2001); Remington: The Science and Practice of Pharmacy, Gennaro, ed., Lippincott Williams & Wilkins (20^{th} ed., 2003); and Pharmaceutical Dosage Forms and Drug Delivery Systems, Ansel et al. (eds.), Lippincott Williams & Wilkins (7^{th} ed., 1999).

The therapeutic formulations can be conveniently presented in unit dosage form and administered in a suitable therapeutic dose. A suitable therapeutic dose can be determined by any of the well known methods such as clinical studies on mammalian species to determine maximum tolerable dose and on normal human subjects to determine safe dosage. Except under certain circumstances when higher dosages may be required, the preferred dosage of an HIV-inhibiting compound usually lies within the range of from about 0.001 to about 1000 mg, more usually from about 0.01 to about 500 mg per day.

The preferred dosage and mode of administration of an HIV-inhibiting compound can vary for different subjects, depending upon factors that can be individually reviewed by the treating physician, such as the condition or conditions to be treated, the choice of composition to be administered, including the particular HIV-inhibiting compound, the age, weight, and response of the individual subject, the severity of the subjects symptoms, and the chosen route of administration. As a general rule, the quantity of an HIV-inhibiting compound administered is the smallest dosage which effectively and reliably prevents or minimizes the conditions of the subjects. Therefore, the above dosage ranges are intended to provide general guidance and support for the teachings herein, but are not intended to limit the scope of the invention.

### EXAMPLES

The following examples are provided to illustrate, but not to limit the present invention.

### Example 1

### General Materials and Methods

Cells lines and maintenance: HeLaCD4βgal cells from Dr. Michael Emerman were obtained through the AIDS Research and Reference Reagent Program, Division of AIDS, NIAID, NIH (Kimpton, J. Virol. 66:2232-9, 1992). The cells were maintained in DMEM supplemented with 10% FBS, 1× Penicillin/Streptomycin/L-glutamine, 0.2 mg/mL G418 and 0.1 mg/mL Hygromycin B. Jurkat cells were maintained in RPMI-1640 supplemented with 10% FBS and 1× Penicillin/Streptomycin/L-glutamine. All cell culture reagents were obtained from Invitrogen.

cDNA screening in HeLaCD4βgal cells: High throughput cDNA retro-transfection of HeLaCD4βgal cells was carried out essentially as described in Chanda et al., Proc. Natl. Acad. Sci. USA 100:12153-8, 2003. Briefly, individual cDNA of a sub-genomic library encompassing 15,000 genes (collection details at http://function.gnf.org), negative control Sport6GFP cDNA and positive control Sport6-Tat plasmid were spotted at 40 ng/well in 55 white opaque 384-well plates (Greiner). A solution of 1% Gene Juice (Novagen) containing 1 μg/mL of an HIV-LTR-Luciferase reporter plasmid (derived by PCR amplification of the LTR sequence of strain HxB2) in serum free Opti-MEM media (Invitrogen) was added to each well (10 μL) using a Multidrop apparatus (Titertek) and complexes were allowed to form for 10 minutes. HeLaCD4βgal cells (1000 cells/30 μL/well in DMEM/10% FBS) were then added and the plates were incubated overnight, followed by the addition of 20 μL of DMEM/10% FBS with 200 ng/mL p24 of HIV-IIIb (Advanced Biotechnologies Inc.). After 72 hours, infection was assessed by measuring luciferase production using Brite Glo (Promega) and reading on the CLIPR apparatus (Molecular Devices). The entire library was run in duplicate. Data for each cDNA was compared to the mean signal of the entire plate, and expressed as the ratio afa/mfa, which is the average fold activation (afa) divided by the adjusted standard deviation of the fold activation (mfa). The mfa penalizes the value for fold activation if the standard deviation between the replicates is high. Hits were reconfirmed after growing up additional cDNA by testing in the original assay and were sequenced to confirm their identity (Eton Bioscience).

cDNA testing of kinase-inactive MLK3: MLK3 and a kinase-inactive mutant of MLK3 (K144R) in pcDNA3.1 vector (Xu et al., Mol. Cell. Biol. 21:4713-24, 2001) were tested alongside the MLK3 Origene collection hit in 12-well plates using a scaled up version of the screen assay. Briefly, 1.28 μg cDNA/0.32 μg LTR-Luc/32 μL was spotted per well followed by 320 μL of 1% gene juice/Optimem and then 6×10⁴ HeLaCD4βgal cells in 1 mL of DMEM/10% FBS. After 24 hours, cultures were infected with 90 ng p24 of HIV-IIIb, incubated for three additional days, and assessed for infection levels using Brite Glo (Promega) and reading on the CLIPR apparatus (Molecular Devices).

siRNA: GL2 luciferase siRNA (catalog #D-001100-01-20), and PITPNα (accession # NM_{—}006224), TRIM28 (accession # NM_{—}005762), IFIT1 (accession # NM_{—}001548), LYZ (accession # NM_{—}000239), CORO1A (accession # NM_{—}007074), and DnaJC14 (accession # NM_{—}032364) Smartpool siRNAs were obtained from Dharmacon. In the case of GL2 siRNA, additional amounts of the same sequence were also obtained from Qiagen. Two siRNA against Tat were synthesized and pooled for use as a positive control for inhibition of HIV infection. MLK3-1 and MLK3-2 siRNA were designed and synthesized, and MLK3-3 siRNA was obtained from Dharmacon (catalog #D-003577-03).

siRNA screening in HeLaCD4βgal cells: The siRNA library was directed against 5000 different genes that have the most potential to be drug targets, with each gene represented by two different siRNAs. siRNA retro-transfection of HeLaCD4βgal cells was carried out essentially as described in Aza-Blanc et al., Molecular Cell 12:627-37, 2003. Briefly, individual siRNAs were spotted at 14 ng/well in white opaque or white clear-bottom 384-well plates (Greiner) containing one siRNA sequence per well. A solution of 2% oligofectamine (Invitrogen) in serum free Opti-MEM media (Invitrogen) was added to each well (10 μL) and complexes were allowed to form for 15-20 minutes. All 384-well dispenses were done using a Multidrop apparatus (Titertek). HeLaCD4βgal cells (1000 cells/30 μL/well in serum free Opti-MEM) were then added and the plates were incubated overnight, followed by the addition of 20 μL of 30% FBS/DMEM with 200 ng/mL of HIV-IIIb (Advanced Biotechnologies Inc.). After 72 hours, infection was assessed by measuring beta-galactosidase production using an equal volume of Gal Screen (Applied Biosystems). All 384-well plate reading was done using the CLIPR apparatus (Molecular Devices). Twelve replicates were run per 384-well plate and the data was expressed as percent inhibition compared to the negative control GL2 siRNA. Cytotoxicity of the siRNA was measured at 96 hours post-transfection by adding equal volume of a 1:4 dilution of Cell Titer Glo (Promega) and reading luminescence on the CLIPR apparatus, with the data again expressed as a percent inhibition compared to GL2.

siRNA validation by western blot: siRNA (800 ng) was spotted in 250 μL of serum free Opti-MEM (Invitrogen) in 6 well plates followed by the addition of 250 μL of 1.5% Lipofectamine 2000 (Invitrogen) in serum free Opti-MEM. Plates were incubated at room temperature for 20 minutes to allow for complex formation. HeLaCD4βgal cells (3×10⁵ in 1.5 mL of serum free Opti-MEM) were then added and incubated overnight followed by the addition of 1 mL of 30% FBS/DMEM. Cells were harvested 72 hours post-transfection by scraping and lysed in cell lysis buffer (20 mM Hepes pH 7.2/10 mM KCl/1 mM EDTA/1% Triton X-100/1× protease inhibitors; Sigma Chemical Co.) for one hour on ice. Total protein concentration of the lysates was measured using the Micro-BCA kit (Promega) and equal protein amounts were loaded onto 4-12% NuPage Bis-Tris gel (Invitrogen) and subjected to electrophoresis as suggested by the manufacturer. Following transfer to nitrocellulose, blots were blocked with 5% nonfat milk in PBST (phosphate buffered saline with 0.05% Tween-20) and then subjected to immunoblotting with the following antibodies; rabbit polyclonal anti-MLK3 and goat-anti-tubulin antibody from Santa Cruz Biotechnology, HRP-conjugated goat-anti-rabbit secondary antibody from Sigma Chemical Co., HRP-conjugated donkey-anti-goat secondary antibody from Promega. All antibodies were used at dilutions suggested by the manufacturer and were diluted in 5% nonfat milk in PBST. Bands were visualized using ECL-plus detection reagent (Amersham).

siRNA transfection of Jurkat T-cells: Jurkat T-cells were washed once in PBS, resuspended in serum free Opti-MEM (Invitrogen) at high density (2.4×10⁸/mL) and 50 μL was added to 1 nmol of siRNA (50 μL of 20 μM) in an 0.2 cm gap cuvette. The mixture was subjected to electroporation using the BioRad Gene Pulser Xcell module using conditions suggested by the manufacturer (140 V, 1000 uF, exponential decay) and then transferred to 12 mL of RPMI supplemented with 10% FBS without antibiotics for 24 hours. Cells were then pelleted, viable cells counted by trypan blue exclusion, and resuspended at a density of 1.7×10⁶/mL in RPMI supplemented with 10% FBS and 1×Pen/Strep/Glutamine. For infection studies, 300 μL of siRNA treated cells were added to wells of a 48 well plate and either 2 ng or 0.5 ng of HIV-IIIb, corresponding to MOIs of 0.0005 and 0.000125 respectively based on viral titer provided by the manufacturer, was added to each well. After three additional days, the cells were harvested, washed 3× in PBS, lysed, and infection was measured by p24 ELISA of the cell lysates. For cytotoxicity, 300 μL of siRNA treated cells were added to wells of a 48 well plate and after 3 days, cell viability was measured using Cell Titer Glo (Promega) and reading on the CLIPR (Molecular Devices) or using Alamar Blue (TREK systems) and reading on the Acquest (LJL Biosystems). To determine siRNA efficacy, cell lysates were prepared and analyzed as in HeLaCD4βgal siRNA validation studies at 72 hours post-electroporation.

### Example 2

### Identification of Novel HIV-Interacting Host Factors from siRNA Screening

We performed a sub-genomic siRNA screen for host proteins that are involved in HIV infection by monitoring expression of a reporter gene under the control of HIV LTR promoter in HeLaCD4Bgal cells (Kimpton et al., J Virol 66:2232-2239, 1992). The HeLa-CD4-Bgal cells were obtained from Dr. Michael Emerman through the AIDS Research and Reference Reagent Program, Division of AIDS, NIAID, NIH. The cells were transfected using a reverse transfection protocol with siRNA against Tat used as a positive control and were challenged with HIV-IIIb 24 hours after transfection (Huang et al., Proc. Nat. Acad. Sci. U.S.A. 101:3456-61, 2004). Infection was allowed to proceed for 3 days to allow for effects on all stages of infection from entry to release and spread throughout the culture to be seen. By monitoring reporter gene expression in the HeLa-CD4-Bgal cells, this system allows one to detect any modulating effect of the siRNAs on HIV infection.

Infection was assessed by measuring the amount of beta-galactosidase produced off the viral LTR promoter using a chemiluminescent substrate. The entire screen was conducted in duplicate and the data was expressed as a ratio of average fold activation (afa) to adjusted standard deviation of the fold activation (mfa), a value that takes into account both the effect of each siRNA and the deviation between the replicates. For those genes whose afa was less than 1 (inhibitors of infection), values were converted to negative fold activation.

### Example 3

### Identification and Characterization of HIV-Interacting Host Factors from cDNA Screening

We performed a high-throughput screen of a cDNA library of 15,000 unique genes to find novel pro-viral factors whose overexpression would lead to enhancement of HIV-IIIb infection. Because of their ease of transfection, we employed HeLaCD4βgal cells for screening and challenged with replication competent HIV-IIIb. Negative control (Sport6-gfp) and positive control (Tat-Sport6) cDNAs were spotted into wells of 384-well plates containing the library cDNA (one gene per well) followed by the addition of transfection reagent solution containing an LTR-luciferase reporter construct responsive to HIV Tat. Cells were added after complex formation, and after 24 hours, each well was infected with HIV-IIIb. Infection was allowed to proceed for 3 days to allow for effects on all stages of infection from entry to release and spread throughout the culture to be observed. Infection was then assessed by measuring the amount of luciferase produced via the viral LTR promoter.

The majority of positive control Tat cDNA wells showed enhancement compared to the negative control and empty wells. The entire screen was conducted in duplicate and the data was expressed as a ratio of average fold activation (afa) to adjusted standard deviation of the fold activation (mfa), a value that takes into account both the effect of each cDNA and the deviation between the replicates. Out of the entire library, 315 (2.1%) genes increased infection with an afa.mfa ≧2, the cutoff chosen for follow-up. Several genes already known to be involved in HIV infection, such as the enhancers S100A12, PP2A regulatory subunit B and nuclear exportin CRM1, were identified as enhancers by the screen, serving as an internal validation. The top 89 enhancers of infection, as determined both by screen performance and literature review, were chosen for follow-up. The hits represented a variety of gene families with the majority being potential druggable targets, including kinases, other enzymes and transcription factors, and from pathways known to be important in HIV infection, including the ubiquitin pathway and RNA processing factors. Each cDNA hit was re-grown at mini-prep scale and tested in the original assay where 19 of the hits reconfirmed. These were then grown at maxi-prep scale, tested in the original assay, and sequenced to ensure proper hit identity. At this stage, 13 sequence-confirmed hits continued to show enhancement of activity compared to the negative control, ranging from 1.7 fold to 8.8 fold over Sport6-GFP negative control (Table 1).

We next investigated whether the strongest cDNA enhancers were essential for HIV replication by depleting the endogenous proteins using siRNA. For 6 of the genes performing ˜3 fold or greater over control we obtained validated siRNA Smartpools and evaluated their effects on HIV-IIIb infection in HeLaCD4βgal cells. Of those tested, siRNA against chaperone DnaJC14, interferon-induced protein with tetratricopeptide repeats 1 (IFIT1) and phosphatidylinositol transfer protein alpha (PITPNα) decreased HIV replication without significant toxicity further strengthening their role in infection, while the others had no effect on viral replication or cell viability (data not shown). Interestingly, one of the genes that both enhanced infection when overexpressed and blocked infection when depleted was the interferon-induced protein IFIT1, which has been shown to be upregulated in a variety of viral infections including Hepatitis C(HCV) as well as in HIV infection of astrocytes. While the biological function of IFIT1 is unknown, it has been reported to be able to interact with Rho/Rac guanine nucleotide exchange factor, and thus may help in the activation of Rho proteins. The idea that HIV may use an interferon-induced protein to help facilitate its replication is intriguing and worth further study. The other two genes with dual activity, the chaperone protein DnaJC14 and PITPNα which is a lipid transport protein involved in vesicle trafficking, also promise to yield further insight into virus biology.

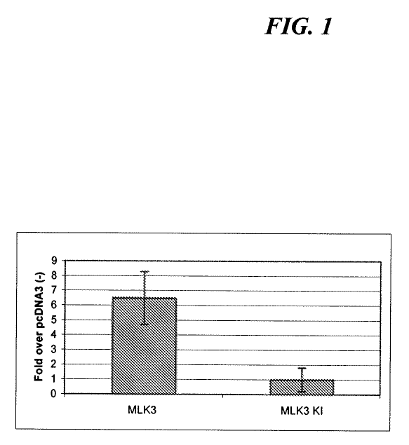

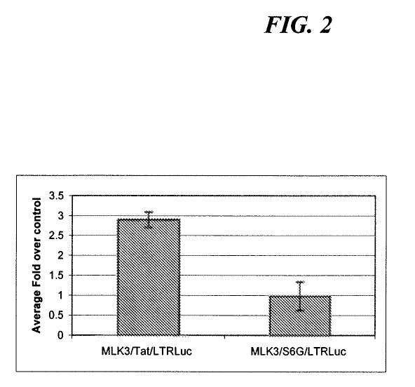

Because of its clear physiologic role and high potential as a therapeutic target, we next focused our efforts on the strong cDNA enhancer mixed lineage kinase 3 (MLK3). This serine/threonine kinase is involved in the activation of downstream Map kinases, including JNK, p38, and ERK, and has been shown to play a critical role in neuronal apoptosis including that mediated by HIV gp120. A compound inhibitor of MLK3, CEP-1347, was in clinical trial for the treatment of neurodegenerative diseases, highlighting its role in neuronal cell death and its applicability as a drug target (Bodner et al., Experimental Neurology 188:246-53, 2004). To address whether the kinase activity of MLK3 was responsible for the enhancement of infection seen using the cDNA, we obtained a kinase-inactive mutant (MLK3 KI) and tested its effects on HIV infection. Unlike the wild type protein, MLK3 KI was unable to increase infection, suggesting that the function of MLK3 is critical for enhancement (FIG. 1). Because of its role in activating the AP-1 complex through JNK activation, we hypothesized that MLK3 could be acting by enhancing HIV transcription via the AP-1 site located in the HIV LTR. To test this, MLK3 was co-transfected with the LTR-luciferase construct either with a Tat expression vector or a control plasmid to assess both Tat-dependent and Tat-independent effects on LTR-mediated transcription. Expression of MLK3 enhanced Tat-dependent transcription by approximately 3-fold over control but had no significant effect on Tat-independent transcription (FIG. 2), suggesting that MLK3 enhances infection through viral specific transcription.

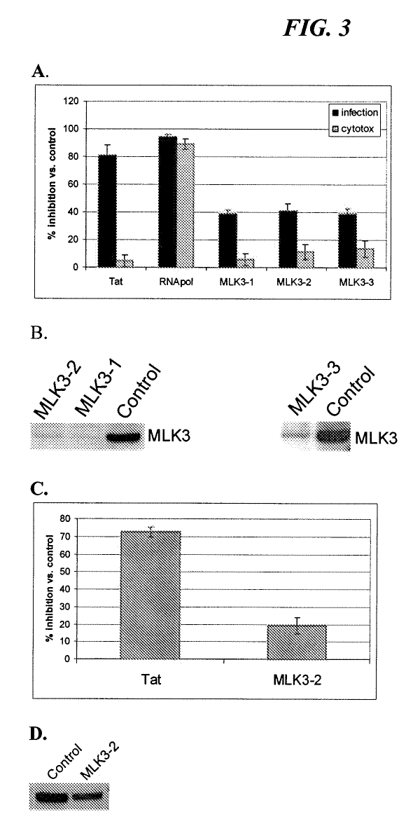

Considering the overexpression data, we finally investigated whether siRNA against MLK3 would inhibit HIV infection. We obtained three unique siRNA sequences targeting MLK3 and evaluated their efficacy against HIV in HeLa-CD4-βgal cells or Jurkat cells. Individual MLK3 siRNAs were transfected into HeLaCD4βgal cells or electroporated into Jurkat cells. After 24 hours, the cells were challenged with HIV-IIIb. For HeLaCD4βgal cells, infection was assessed after 3 additional days by measuring the beta-galactosidase produced from the stable LTR-βgal reporter within the cells using a chemiluminescent substrate (Gal Screen). For Jurkat cells, input virus was removed 24 hours after infection, and supernatants were collected after an additional 48 hours and tested for levels of p24 by ELISA. Cytotoxicity of each siRNA was also determined by using Cell Titer Glo in parallel uninfected cultures. The results indicate that all three siRNA were effective at depleting MLK3 protein (FIG. 3B) and decreased HIV infection by approximately 40% (FIG. 3A) in HeLaCD4βgal cells. Consistent with its proposed mechanism of affecting Tat-dependent transcription, MLK3 depletion had no effect on levels of early reverse transcripts or integrated provirus (data not shown). In addition, the MLK3 siRNAs also decreased HIV-IIIb infection of Jurkat cells, although to a lesser extent than that seen in the HeLa-CD4-Bgal cells (FIG. 3C). Western blot analysis showed that the amount of protein depletion in the Jurkat cells was lower than that seen in the HeLaCD4Bgal cells, thus explaining the lower level of inhibition seen (FIG. 3D).

TABLE 1
HIV-IIIb HeLa-CD4-Bgal screen: general results:

| Range of hits | −8.9 to +4.9 fold afa.mfa |
|---|---|
| Afa.mfa range | # of hits (10k total library) |

Proviral factors (inhibition with siRNA inhibits
infection):

| | |
|---|---|
| −8.9 to −5 | 61 (0.6%) |
| −5 to −3 | 280 (2.8%) |
| −3 to −2 | 741 (7.41%) |

Anti-viral factors (inhibition with siRNA promotes
infection):

| | |
|---|---|
| +2 to +3 | 365 (3.65%) |
| +3 to +4.9 | 53 (0.53%) |

TABLE 2
Confirmed HIV-interacting host factors identified by siRNA screening

| | | | | Reconfirmation | |
|---|---|---|---|---|---|
| | | | | results | |
| | | | | % | |
| | | | orig | infection | % |
| Acc. No. | Gene hit | Gene | afa.mfa | inhibition | cytotoxicity |
| NM_000208 | INSR | insulin receptor | −3.22 | 81.45 | 7.88 |
| NM_013278 | IL17C | interleukin 17C | −4.05 | 71.07 | 14.86 |
| NM_004736 | XPR1 | xenotropic and polytropic | −4.94 | 70.71 | 15.92 |
| | | retrovirus receptor | | | |
| NM_006978 | ZNF183 | zinc finger protein 183 (RING | −3.68 | 87.57 | 24.95 |
| | | finger, C3HC4 type) | | | |
| NM_000099 | CST3 | cystatin C (amyloid angiopathy | −3.49 | 78.42 | 25.83 |
| | | and cerebral hemorrhage) | | | |
| NM_002529 | NTRK1 | neurotrophic tyrosine kinase, | −3.49 | 61.21 | 26.95 |
| | | receptor, type 1 | | | |
| NM_001752 | CAT | catalase | −4.32 | 72.57 | 27.70 |
| NM_178432 | CCRK | cell cycle related kinase | −3.02 | 83.89 | 30.46 |
| NM_003352 | UBL1 | ubiquitin-like 1 (sentrin) | −2.59 | 83.33 | 31.01 |
| NM_001334 | CTSO | cathepsin O | −2.57 | 71.93 | 32.49 |
| NM_032875 | MGC15482 | F-box protein FBL2 | −5.94 | 95.24 | 33.33 |
| NM_020341 | PAK7 | p21(CDKN1A)-activated kinase 7 | −2.52 | 68.61 | 36.04 |
| NM_000924 | PDE1B | phosphodiesterase 1B, | −3.84 | 91.01 | 37.71 |
| | | calmodulin-dependent | | | |
| NM_014339 | IL17R | interleukin 17 receptor | −3.32 | 87.07 | 38.15 |
| NM_014215 | INSRR | insulin receptor-related receptor | −4.91 | 88.16 | 38.51 |
| NM_003877 | SOCS2 | suppressor of cytokine signaling 2 | −3.29 | 67.78 | 38.74 |
| NM_052867 | VGCNL1 | voltage gated channel like 1 | −3.08 | 85.22 | 39.93 |
| NM_003954 | MAP3K14 | mitogen-activated protein kinase | −3.01 | 76.62 | 40.15 |
| | | kinase kinase 14 | | | |
| NM_000211 | ITGB2 | integrin, beta 2 (antigen CD18 | −3.12 | 74.21 | 40.31 |
| | | (p95), lymphocyte function- | | | |
| | | associated antigen 1; macrophage | | | |
| | | antigen 1 (mac-1) beta subunit) | | | |
| NM_005056 | RBBP2 | retinoblastoma binding protein 2 | −3.18 | 76.76 | 41.11 |
| NM_006748 | SLA | Src-like-adaptor | −5.98 | 92.69 | 41.87 |
| NM_001315 | MAPK14 | mitogen-activated protein kinase | −4.07 | 82.06 | 42.00 |
| | | 14 | | | |
| NM_023006 | KLK15 | kallikrein 15 | −3.22 | 89.76 | 44.88 |
| NM_006079 | CITED2 | Cbp/p300-interacting | −2.93 | 78.67 | 45.05 |
| | | transactivator, with Glu/Asp-rich | | | |
| | | carboxy-terminal domain, 2 | | | |
| NM_003809 | TNFSF12 | tumor necrosis factor (ligand) | −3.22 | 88.03 | 46.58 |
| | | superfamily, member 12 | | | |
| NM_002788 | PSMA3 | proteasome (prosome, | −3.47 | 81.45 | 46.87 |
| | | macropain) subunit, alpha type, 3 | | | |
| NM_021079 | NMT1 | N-myristoyltransferase 1 | −3.39 | 85.07 | 47.87 |
| NM_002214 | ITGB8 | integrin, beta 8 | −3.19 | 86.80 | 48.12 |
| NM_000352 | ABCC8 | ATP-binding cassette, sub-family | −3.08 | 90.45 | 50.11 |
| | | C (CFTR/MRP), member 8 | | | |
| NM_000958 | PTGER4 | prostaglandin E receptor 4 | −3.69 | 93.57 | 50.13 |
| | | (subtype EP4) | | | |
| NM_002208 | ITGAE | integrin, alpha E (antigen | −3.33 | 91.89 | 50.38 |
| | | CD103, human mucosal | | | |
| | | lymphocyte antigen 1; alpha | | | |
| | | polypeptide) | | | |
| NM_007049 | BTN2A1 | butyrophilin, subfamily 2, | 4 | 87.38 | 50.74 |
| | | member A1 | | | |
| NM_003331 | TYK2 | tyrosine kinase 2 | −5.12 | 89.69 | 51.06 |
| NM_001244 | | TNFSF8 | −3.28 | 91.04 | 51.92 |

TABLE 3
Confirmed HIV-interacting host factors identified by cDNA screening

| | | | Original | Reconfirmation |
|---|---|---|---|---|
| | | | hit | Fold over |
| Gene Hit | Accession # | Symbol | afa.mfa^{a} | control^{b} |
| Phosphotidylinositol transfer | NM_006224 | PITPNα | 2.77 | 2.95 |
| protein alpha | | | | |
| Extracellular link domain | NM_006691 | XLKD1 | 2.60 | 2.7 |
| containing 1 | | | | |
| Tripartite motif-containing 28 | NM_005762 | TRIM28 | 2.67 | 5.8 |
| Lysozyme | NM_000239 | LYZ | 2.67 | 8.6 |
| Interferon-induced protein with | NM_001548 | IFIT1 | 4.90 | 3.5 |
| tetratricopeptide repeats 1 | | | | |
| Coronin, actin binding protein, 1A | NM_007074 | CORO1A | 2.90 | 2.92 |
| Clone PP902 | AF218032 | | 2.47 | 2.4 |
| DnaJC14 protein | NM_032364 | | 5.70 | 5.5 |
| Aldehyde dehydrogenase 3 family, | NM_000691 | ALDH3A1 | 2.07 | 1.70 |
| memberA1 | | | | |
| SET domain-containing protein 8 | AY102937 | SET8 | 2.13 | 2.2 |
| Mitogen-activated protein kinase | NM_002419 | MAP3K11, | 4.60 | 8.80 |
| kinase kinase 11 | | MLK3 | | |
| KIAA0247 gene product | NM_014734 | | 3.90 | 2.01 |
| Dual specificity phosphatase 18 | XM_038481 | DUSP18 | 2.99 | 2.10 |

^{a}Average fold activation (afa) divided by the adjusted standard deviation of the fold activation (mfa), which accounts for both the effect of each cDNA and the deviation between the replicates.
^{b}Negative control Sport6gfp, same as used in screen.

### Example 4

### HIV-Interacting Host Factors Identified by Yeast Two-Hybrid Screening

A yeast two-hybrid screening was performed to identify novel interacting host cell factors encoded in a human leukocyte cDNA library. Assays of protein-protein interaction in yeast were done with GALA and LexA fusion proteins. Vpr cDNA was amplified by PCR using Vpr-specific primers. The cDNA was inserted into the LexA DBD expression vector pSLANS. pSLANS is a modified version of pBTM116 (Bartel et al Biotechniques 14: 920-924, 1993). It was modified to accept NotI inserts and to place gly4-ser-gly4-ser between LexA and the bait. The cDNA encoding the LexA DBD-Vpr fusion protein was transformed into the L40 MATa yeast strain. The cDNAs encoding the Gal4 AD—leukocyte cDNA fusion proteins were transformed into the yeast strain 540 MATα. The two yeast strains were mated and transformants containing both plasmids were selected in THUKL-deficient synthetic media, and protein interactions were analyzed by a β-galactosidase filter assay.

Approximately two million diploid transformants were screened and numerous positive candidates were isolated. Positive colonies were assayed for β-galactosidase activity using a filter lift assay for further confirmation of the protein-protein interaction. The screening hits, as listed in Table 4, were identified by sequencing the candidate cDNA clones and comparison to the Genbank database.

TABLE 4
HIV-interacting host factors identified by yeast two-hybrid screening

| Accession No. | Gen Name | Description |
|---|---|---|
| AK026228.1 | FLJ22575 | Homo sapiens cDNA: FLJ22575 fis, clone HSI02453, highly |
| | | similar to AF155105 Homo sapiens putative zinc finger protein |
| | | NY-REN-34 antigen mRNA |
| AC002299.1 | HS Chrom 16 | Homo sapiens Chromosome 16 BAC clone CIT987-SKA- |
| | BAC clone | 113A6 ~complete genomic sequence |
| AB033001 | KIAA1175 | Homo sapiens mRNA for KIAA1175 protein, partial cds |
| NM_002211 | ITGB1 | Homo sapiens integrin, beta 1 (fibronectin receptor, beta |
| | | polypeptide, antigen CD29 includes MDF2, MSK12) (ITGB1), |
| | | transcript variant 1A |
| AF223391.1 | CACNA1E | Homo sapiens calcium channel alpha1E subunit (CACNA1E) |
| | | gene, exons 7-49, and partial cds, alternatively spliced |
| NM_001456 | FLNA | Homo sapiens filamin A, alpha (actin binding protein 280) |
| NM_003775.1 | EDG6 | Homo sapiens endothelial differentiation, G-protein-coupled |
| | | receptor 6 (EDG6), |
| AL390127.1 | DKFZp761P06121 | Homo sapiens mRNA; cDNA DKFZp761P06121 (from clone |
| | | DKFZp761P06121) |
| NM_016142.1 | HSD17B12 | Homo sapiens hydroxysteroid (17-beta) dehydrogenase 12 |
| | | (HSD17B12) |
| M27288.1 | Oncostatin M, | Human oncostatin M gene, exon 3 |
| NM_004247.1 | EFTUD2 | Homo sapiens elongation factor Tu GTP binding domain |
| | | containing 2 (EFTUD2) |
| M31951.1 | PRF1 | Human perforin (PRF1) gene, complete cds |
| NM_012227.1 | GTPBP6 | Homo sapiens GTP binding protein 6 (putative) (GTPBP6) |

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, ATCC deposits, patents and patent applications cited herein are hereby expressly incorporated by reference in their entirety and for all purposes as if each is individually so denoted.

We claim:

1. A method for identifying an agent that inhibits HIV infection, the method comprising:
(a) screening test compounds to identify one or more modulating compounds that down-regulate a biological activity or expression level of an HIV-interacting host factor encoded by a polynucleotide selected from the members listed in Tables 2-4; and
(b) testing the modulating compounds for ability to inhibit HIV infection.

2. The method of claim 1, wherein the HIV-interacting host factor is selected from the group consisting of tetratricopeptide repeats 1 (IFIT1), phosphatidylinositol transfer protein alpha (PITPNα), and mixed lineage kinase 3 (MLK3).

3. The method of claim 2, wherein the HIV-interacting host factor is MLK3, and the test compounds are screened for ability to inhibit the kinase activity of MLK3 or its expression.

4. The method of claim 1, wherein the ability to inhibit HIV infection by the modulating compounds is examined by monitoring expression of a reporter gene under the control of HIV LTR promoter in an HIV-infected cell.

5. The method of claim 4, wherein the cell is HeLa-CD4-Bgal.

6. The method of claim 4, wherein the reporter gene is a beta-galactosidase gene.

7. The method of claim 4, wherein the cell is infected by HIV-IIIb.

8. The method of claim 1, wherein the ability to inhibit HIV-1 infection by a modulating compound is examined by comparing HIV replication in an engineered HIV permissive cell that has been contacted with the modulating compound with HIV replication in a control cell that has not been contacted with the compound.

9. The method of claim 8, wherein the HIV permissive cell is HeLa-T4-βGal HIV cell.

10. The method of claim 8, wherein HIV replication is monitored via a p24 antigen ELISA assay or a reverse transcriptase activity assay.

11. The method of claim 1, wherein the ability to inhibit HIV infection by the compound is examined by comparing pseudovirus production in a host cell treated with the compound with pseudovirus production in a control host cell that has not been treated with the compound.

12. The method of claim 11, wherein the host cell is 293T HEK cell.

13. The method of claim 11, wherein the host cell is transfected with pseudovirus plasmids which produce HIV pseudovirus in the cell.

14. The method of claim 1, wherein the HIV-interacting host factor is an enzyme, and the biological activity assayed is its enzymatic activity.

15. The method of claim 13, wherein the enzyme is a kinase.

16. The method of claim 15, wherein the kinase is MLK3.

17. A method for inhibiting HIV infection in a subject, comprising administering to the subject a pharmaceutical composition which comprises an effective amount of a compound that inhibits a biological activity or expression of an HIV-interacting host factor encoded by a polynucleotide selected from the members listed in Tables 2-4.

18. The method of claim 17, wherein the HIV-interacting host factor is selected from the group consisting of tetratricopeptide repeats 1 (IFIT1), phosphatidylinositol transfer protein alpha (PITPNα), and mixed lineage kinase 3 (MLK3).

19. The method of claim 17, wherein the HIV-interacting host factor is MLK3, and the compound inhibits the kinase activity of MLK3.

20. The method of claim 19, wherein the compound is K252a or CEP1347.
